(19) United States
(12) Patent Application Publication
(10) Pub. No.: US 2005/0175724 A1
(43) Pub. Date: Aug. 11, 2005

(54) Composition for modulating a physiological reaction or inducing an immune response

(76) Inventor: Grant William Vandenberg, Saint-Augustin-de Desmauces (CA)

Correspondence Address: OGILVY RENAULT LLP, 1981 MCGILL COLLEGE AVENUE, SUITE 1600, MONTREAL, QC, H3A2Y3, CA

(21) Appl. No.: 10/509,333

(22) PCT Filed: Apr. 8, 2003
(86) PCT No.: PCT/CA03/00523

(30) Foreign Application Priority Data
Apr. 8, 2002 (US) 60/370220

(51) Int. Cl. A61K 35/78; A61K 38/21; A61K 38/43; A61K 39/38

(52) U.S. Cl. 424/757; 514/2; 424/85.4; 424/94.1; 424/184.1; 424/85.1; 424/85.2

(57) ABSTRACT

The present invention relates to a new composition, uses and method for modulating a physiological reaction or inducing an immune response after oral administration to a human or an animal of a physiologically active agent comprising (at least one) neutralizing agents to increase pH in the digestive system to prevent denaturation, (one) inhibitors of digestive enzymes to substantially prevent enzymatic digestion, and at least (one) uptake-increasing agents which increases intestinal absorption of a physiologically active agent, a drug and/or a nutrient.

### BACKGROUND OF THE INVENTION

(a) Field of the invention

The present invention relates to a composition and a method for oral administration of physiological active products and intestinal delivery thereof. The physiological active products administered by the method according to the present invention allow to achieve a better systemic delivery and immunologic induction, and has demonstrated improved nutritional, nutraceutical, and therapeutic capacities.

(b) Description of Prior Art

The intensification of fish farming and increased densities involve a substantial stress which, as in other captive livestock species, has resulted in outbreaks of major diseases and related mortalities. Consequently, this led to a widespread use of antibiotics, as both a prophylactic and a treatment measure. However, the development of antibiotic-resistant bacteria and the accumulation of antibiotics in the environment and in the flesh of fish, underscored the necessity of a vaccination alternative. Indeed, in recent years much effort has been dedicated to vaccine development and the use of vaccines in fish farming has become popular. As in all vertebrates, the specific immune response in fish is composed of humoral immunity and cell-mediated immunity. Upon exposure to an antigen, B-lymphocytes differentiate into plasma cells, which produce specific antibodies, and into memory cells. The T-cells proliferate into long-lived helper memory cells. In contrast with the immune systems of higher vertebrates, fish appears to produce a single dominant class of immunoglobulin, similar to the IgM of mammals.

The T-lymphocytes are also responsible for the cell-mediated response. On primary antigen stimulation they differentiate into killing cells, lymphokine-producing cells and suppressor cells. The cell-mediated immune response in fish involves also macrophages, which are the fish's main line of non-specific defense. In contrast with terrestrial livestock, vaccination in fish is complicated by their aquatic environment. Individual injections of vaccines are labor-intensive and stressful, since fish have to be removed from water and anesthetized prior to injection. It has been demonstrated that certain antigens can induce an immune response in fish when they are introduced into the water that contain the fish (i.e. immersion vaccination). However, the degree and duration of immune protection induced by immersion have been variable. The uptake of Vibriosis vaccine from water into rainbow trout is low, and is limited to 0.01-0.2% of the initial vaccine bath concentration. Similarly, rainbow trout immersed in high concentrations of a Furunculosis vaccine takes up only very small proportions of the antigen, even when immersed for periods of up to 4 hours. When fish are injected with vaccines, the protective antigens can be localized in the spleen and kidney, which are the main organs of phagocytic filtration and the main sites for protective immunity. However, after immersion of fish in the vaccine, the antigens are almost exclusively located on the outer surfaces, skin and gills. In fact, whereas the injection of vaccines induces a systemic immune response (in spleen and kidney), it is believed that the immersion technique induces mainly an integumentary immune response (in the mucous membranes of skin, gills and gut). This explains the findings that for all studied vaccines, the injection of antigens provides far better immune protection than does the immersion protocol. However, in view of the relative simplicity of the immersion procedures as compared to injections, it is considered in the industry that immersion of fish in a solution containing high concentrations of the vaccine results in sufficient protection against certain diseases (e.g. enteric red mouth disease and in some species vibriosis). Nevertheless, in the case of other diseases (e.g. furunculosis and in some species vibriosis), fish have to be individually injected with the vaccine. Yet for other major diseases, such as bacterial kidney disease (BKD), efforts to develop vaccines have been unsuccessful. Oral administration of vaccines has been extensively studied in recent years. However, despite the initial promise of this method (i.e. being completely non-stressful to the fish), oral vaccination was found to confer only a very limited level of immune protection. In some cases, oral administration of antigens even resulted in suppression of the immune response.

In all cases of vaccination in fish, longevity of the immune protection is a major concern. Only rarely is the duration of the protection induced by a single vaccination longer than one year, while rearing time to harvest in most farmed fish is much longer, exceeding two years. Consequently, many of the available fish vaccines are of limited efficiency. Booster vaccinations of large fish, usually by injection, are required to improve the duration of protection. This involves considerable labor, handling, and stress to the fish, which results in mortalities. No delivery systems are currently used in fish to prolong bio-availability of antigens. In addition, a number of potential adjuvants that may have increased the degree of immune protection, cannot be used in commercial practice because they cause severe infections or are toxic.

As is the case in humans, a major problem which impedes the ability to optimally immunize all fish against common diseases is the requirement to deliver multiple doses of the vaccines in order to maintain protection. Current immunization schemes produce episodic stimulation of the immune system because repeated applications of the vaccine have been the only practical method for delivering vaccines. As a result, intensive research has been recently carried out in mammals with the aim of developing single-dose, controlled-release formulations that will efficiently induce a protective primary response of long duration. Based on polymer design, the release pattern of the antigen is either continuous or comprises an initial prolonged release of the antigen intended to prime the immune response, and a delayed release for boosting it. The carrier polymers which are non-toxic and biocompatible, in addition to their role in obtaining optimal release rates as delivery systems are known to have strong adjuvant, non-specific stimulatory effects on the defense mechanisms.

While similar systems have been proposed for applications to permit more effective vaccination of domestic animals and fish, little work has been done to evaluate this strategy in aquatic species. While incorporation of fish vaccines in polymer systems would likely provide sustained vaccine release and improved protection, important physiological differences between species along with disparate conditions (e.g. water temperature) would likely affect polymer function, and would require detailed characterization between species and culture conditions. Furthermore, polymer-mediated sustained vaccine release requires injection, therefore, on a practical level, this strategy may offer relatively limited advantage over non-encapsulated vaccines for aquatic species.

While fish lack some of the specialized cellular and tissue components of the gut associated lymphoid tissues (GALT) of mammals, there is considerable evidence for the ability of enterocytes, especially in the hind gut segment, to take up antigens and translocate them to macrophages and lymphocytes in the lamina propria and, under certain circumstances, to systemic lymphoid organs, i.e. kidney and spleen. Current evidence from carp suggests that oral delivery of antigens stimulates antibody production in the gut, gill and skin, but not in the kidney and blood, while parenteral injection of antigen stimulates the systemic compartment and not the mucosal compartment.

However, in salmonids, it is suggested that oral immunization stimulates only low responses in the gill, gut and skin, while injection stimulates both systemic and mucosal responses and stimulates the latter more effectively than oral immunization.

Thus, while there is evidence of a common mucosal immune system in fish, there seems to be some species variation in the extent of its compartmentalization. There is also species variation in the nature of antigen uptake by the enterocytes. In carp (Cyprinus carpio), only soluble antigens are taken up (by pinocytosis), while in salmonids, whole bacterial cells, as well as soluble antigens, can be taken up. Furthermore, exposure of the antigen to conditions in the anterior gut, while not necessarily preventing uptake by hind gut enterocytes, may affect antigen translocation to the systemic compartment. Present information, while still very incomplete, indicates that orally delivered antigen must be protected from digestion and other forms of modification in the anterior intestine and be delivered to the hind gut enterocytes in a form which can be taken up by these cells and translocated in a strongly immunogenic form to the systemic immune compartment.

There is, therefore, a pressing need for a new, efficient, cost effective and non-invasive method of administration to humans and animals of a composition containing nutritional and therapeutic agents, particularly peptides, which are otherwise unsuitable for oral administration.

### SUMMARY OF THE INVENTION

An object of the present invention is to provide a composition for modulating at least one physiological reaction or inducing an immune response in human or animal after oral administration, including mammals, birds, insects, crustaceans, amphibians, reptiles and fish, for intestinal delivery of a physiologically active agent comprising, a neutralizing agent that increases pH in the animal digestive system to prevent chemical denaturation, an inhibitor of digestive enzymes to prevent enzymatic digestion of the active agents, and an uptake increasing agent which increases intestinal absorption of a physiologically active agent. The invention is also based upon the finding that the combination of the three agents provides additive and synergistic intestinal delivery and uptake when used concurrently.

Another object of the present invention is to provide a method for treating intestinal microbial infections in an animal, which comprises administrating a sufficient amount of the composition of the present invention, wherein the physiologically active agent is an antimicrobial agent.

In accordance with the present invention, there is provided a composition for oral administration to an animal for intentional delivery of a physiologically action agent or antigen, the composition of the present invention comprising at least one neutralizing agent at concentration between about 1% to 60% w/w, an enzymatic inhibitor at concentration between about 1% to 50% w/w, and an uptake increasing agent at concentration between about 0.1% to 50% w/w.

The composition according to the present invention, may also comprise a physiologically active agent selected from the group consisting of, but not limited to, therapeutical agents, nutritional products, mucopolysaccharides, lipids, carbohydrates, steroids, hormones, growth hormones (GH), growth hormone releasing hormones (GHRH), epithelial growth factors, vascular endothelial growth and permeability factor (VEGPF), nerve growth factors, cytokines, interleukins, interferons, GMCSF, hormone-like products, neurological factors, neurotropic factors, neurotransmitters, neuromodulators, enzymes, antibodies, peptides, proteic fragments, vaccines, adjuvants, antigens, immune stimulating or inhibiting factors, heomatopoietic factors, anti-cancer products, anti-inflammatory agents, anti-parasitic compounds, anti-microbial agents, nucleic acid fragments, plasmid DNA vectors, cell proliferation inhibitors or activators, cell differentiating factors, blood coagulation factors, immunoglobulins, negative selective markers or “suicide” agents, toxic compounds, anti-angiogenic agents, polypeptides, anti-cancer agents, acid production drugs, and histamine H2-receptor antagonists.

The composition of the present invention may comprise a neutralizing agent that is in amount sufficient to neutralize acidic degradation of the digestive system of the host animal and allow delivery of a physiologically active agent to the animal intestine, where the neutralizing agent may be selected from the group consisting of anti-acids, sodium bicarbonate, sodium carbonate, sodium citrate, sodium hydrogencarbonate, calcium phosphate, calcium carbonate, magnesium salts, magnesium carbonate, magnesium trisilicate, magnesium hydroxide, magnesium phosphate, magnesium oxide, bismuth subcarbonate, and combinations thereof.

In addition, the composition of the present invention may comprise a neutralizing agent which consists of at least one of sodium carbonate at a concentration of 10% to 20% w/w, and calcium carbonate at concentration of 10% to 20% w/w of the composition.

According to the present invention, there is provided a composition which comprises at least one enzyme inhibitor in an amount sufficient to substantially inhibit the degradation of a physiologically active agent by digestive enzymes in the digestive system of a human or an animal and allow delivery of this physiologically active agent into the intestine of the human or the animal.

The inhibitor of digestive enzymes may be selected from the group consisting of, but not limited to, anti-proteases, egg albumin, plant-derived inhibitors from oilseeds, soybeans, kidney beans, faba beans, rice bran, wheat bran, ethylenediamine tetraacetate, alpha-1-antitrypsin, albumin, ovalbumin, and proteasomes.

The composition according to the present invention may comprise pepsin inhibitors, enteropeptidase inhibitors, and/or albumin at a concentration between 1% to 20% w/w.

The composition of the present invention may comprise an uptake increasing agent which may consist of bile salts, saponins, deoxycholate, sodium salicylate, sodium lauryl sulfate, oleic acid, linoleic acid, monoolein, lecithin, lysolecithin, polyoxyethylene sorbitan esters, p-t-octylphenoxypolyoxyethylene, N-lauryl-μ-D-maltopyranoside, 1-dodecylazacycloheptane-2-azone, and phospholipids.

The uptake-increasing agent may be sodium deoxycholate at a concentration between 0.01% to 10%.

The composition of the present invention may further comprise at least one additional ingredient selected from the group consisting of ethylenediamine tetraacetate, preservatives, antioxidants, colorants binders, tracers, one or more sweeteners, surfactants, unmoulding agents, flavouring agents, meals, beans, yeast, brewer yeast, mineral oil, vegetable oil, animal oil, lubricants, ointment, and combinations thereof.

Another object of the present invention is that physiologically active agent when delivered into the human or the animal intestine may be absorbed by the intestine for systemic delivery, or to have an effective physiological effect on intestinal wall.

Also, the composition according to the present invention may allow for a physiologically active agent when delivered into a human or an animal intestine to have a physiological effect into the content of the intestine. This application may further be used to stimulate food transit throughout the gut, or to treat infectious diseases.

In accordance with the present invention, the physiologically active agent is capable of inducing mucosal immunity or systemic immune reaction in the host human or animal against mucosal infectious diseases. There is provided a method of immunization of a host against mucosal microorganisms which comprises orally administering to the host an immunizing amount of microbial surface protein in the form of killed whole microorganisms, a lysate of microorganisms or an isolated antigen or an immunologic fragment thereof.

The present invention further provides a composition for oral administration to a host, preferably for administration into the gut (stomach, digestive tract) of a host to confer protection or elicit an immune response against microbial infections.

According to the present invention, there is provided a method of treating intestinal microbial infections in an animal, which comprises administering the composition of the present invention comprising an anti-microbial agent in an amount sufficient for therapeutic effectiveness.

The microbial infections may be caused by microorganisms selected from the group consisting of, but not limited to, bacteria, fungi, mushrooms, yeasts, viruses, Staphylococci, Streptococci, Micrococci, Peptococci, Peptostreptococci, Enterococci, Bacillus, Clostridium, Lactobacillus, Listeria, Erysipelothrix, Propionibacterium, Eubacterium, Corynobacterium, Mycoplasma, Ureaplasma, Streptomyces, Haemophilus, Neisseria, Eikenellus, Moraxellus, Actinobacillus, Pasteurella, Bacteroides, Fusobacteria, Prevotella, Porphyromonas, Veillonella, Treponema, Mitsuokella, Capnocytophaga, Campylobacter, Klebsiella, Chlamydia, Furonculosis, and coliforms.

The antimicrobial agent used to treated microbial infections may be selected from the group consisting of, but not limited to, antibiotics, bacteriocins, lantibiotics, probiotics, antifungics, antimycotics, antiparasitics, aminoglycosides, vancomycin, rifampin, lincomycin, chloramphenicol, fluoroquinol, penicillin, beta-lactams, amoxicillin, ampicillin, aziocillin, carbenicillin, mezlocillin, nafcillin, oxacillin, piperacillin, ticarcillin, ceftazidime, ceftizoxime, ceftriaxone, cefuroxime, cephalexin, cephalothin, imipenen, aztreonam, gentamicin, netilmicin, tobramycin, tetracyclines, sulfonamides, macrolides, erythromicin, clarithromcin, azithromycin, polymyxin B, clindamycin antibiotic, and combinations thereof.

The invention is also to provide a method of systemic delivery, which comprises oral administration to an animal of a therapeutical agent for treating a health disorder of the animal, which may further comprises an acceptable pharmaceutical carrier.

The composition of the present invention can be used in the manufacture of drugs or foods.

There is also provided according to the present invention a method for enhancing intestinal uptake in human or animal, which comprises orally administering a physiologically effective amount of a physiologically active agent.

Another object of the present invention is to provide a method for modulating a physiological reaction or inducing an immune response in a human or an animal comprising orally administrating a sufficient amount of a composition of the present invention defined herein.

For the purpose of the present invention the following terms are defined below.

The term “therapeutic agent” is used in a generic sense and includes treating agents, prophylactic agents, replacement agents, and antimicrobial agents.

The term “common mucosal immune system” refers to the fact that immunization at any mucosal site can elicit an immune response at all other mucosal sites.

The terms “protein”, “peptide” and “polypeptide” refer to both the naturally occurring chemical entities and the structurally similar bioactive equivalents derived from either endogenous, exogenous, or synthetic sources and is used to mean polymers of amino acids linked together by an amide type linkage known as a peptide bond.

The term “structurally similar bioactive equivalent” means a polypeptide with an amino acid sequence which, although not identical to that of the naturally occurring peptide, is sufficiently similar in structure to produce substantially equivalent therapeutic effects on the subject to that produced by the natural peptide itself.

The term “therapeutically effective amount” of a medicament means a sufficient amount of the compound to obtain the intended therapeutic benefit, at a reasonable benefit/risk ratio applicable to any medical treatment. It will be understood, however, that the total daily usage of the medicaments and compositions of the present invention will be decided by the attending physician within the scope of sound medical judgement. The specific therapeutically effective dose level for any particular patient will depend upon a variety of factors including the disorder being treated and the severity of the disorder; activity of the compound employed; the specific composition employed; the age, body weight, general health, sex and diet of the patient; the time of administration, route of administration and rate of excretion of the specific compound employed; the duration of the treatment; drugs used in combination or coincidental with the specific compound employed; and like factors well known in the medical arts. For example, it is well within the skill of the art to start at doses lower than required to achieve the desired therapeutic effect and to gradually increase the dosage until the desired effect is achieved.

### BRIEF DESCRIPTION OF THE DRAWINGS

FIG. 1 illustrates the influence of increasing sodium deoxycholate (g sodium deoxycholate/kg bypass cocktail) on weight gain in rainbow trout in all tanks;

FIG. 2 illustrates the influence of increasing sodium deoxycholate (g sodium deoxycholate/kg bypass cocktail) on weight gain in rainbow trout in extreme tanks;

FIG. 3 illustrates the percentage increase in brook trout weight in bST-supplemented bypass cocktail with increasing levels of sodium deoxycholate;

FIG. 4 illustrates fish weight gain of control and injected fish;

FIG. 5 illustrates the inhibition curve for freeze-dried ovalbumin of the composition of the present invention;

FIG. 6 illustrates the inhibition curve for red kidney beans of the composition of the present invention;

FIG. 7 illustrates the inhibition curve for soybeans of the composition of the present invention;

FIG. 8 illustrates the inhibition curve for faba beans of the composition of the present invention;

FIG. 9 illustrates the inhibition curve for EDTA of the composition of the present invention;

FIG. 10 illustrates the inhibition curve for wheat bran of the composition of the present invention;

FIG. 11 illustrates the inhibition curve for spray-dried ovalbumin of the composition of the present invention;

FIG. 12 illustrates the inhibition curve for combined ingredients of the composition of the present invention;

FIG. 13 illustrates the standard curve of HRP in the plasma of rainbow trout;

FIG. 14 illustrates the effect of the composition of the present invention on HRP uptake;

FIG. 15. illustrates intact HRP absorbed into the general circulation of a fish; and

FIG. 16 illustrates antibody levels after oral immunization of fish.

FIG. 17 illustrates the survival of fish following different types of vaccine administration.

FIG. 18 illustrates the mortalities of fish following different types of vaccine administration.

FIG. 19 illustrates antibody levels of boosted fish after oral immunization.

### DETAILED DESCRIPTION OF THE INVENTION

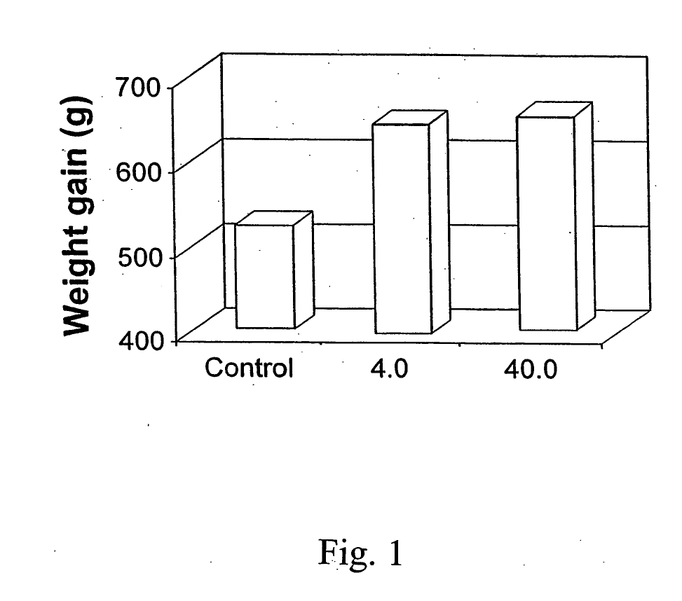

The present invention provides evidence that by using an innovative vaccine composition, it is possible to transport a bioactive protein past the stomach of fish, as for example but not limited to, rainbow trout, whereby the protein is absorbed (FIG. 1).

The effectiveness of using the vaccine composition to deliver vaccines orally in order to overcome current production constraints facing the aquaculture industry is hereby clearly demonstrated. The present invention relates to the administration of therapeutic proteins and polypeptides in oral dosage form. The invention provides increased absorption through the GI tract and greatly improved bioavailability of the proteins/peptides as compared to that of the prior art formulations. The invention is useful both in human and veterinary nutrition, therapy and treatment. As used herein and in the appended claims, the term “polypeptide” encompasses within its scope proteins and peptides as well as polypeptides.

The compounds and compositions of the subject invention are useful for administering biologically or chemically active agents to any animals such as birds, fish, crustaceans, amphibians, reptiles, mammals (such as primates and particularly humans), and insects. The system is particularly advantageous for delivering physiologically, biologically or chemically active agents which would otherwise be degraded or rendered less effective by conditions encountered before the active agent reaches its target zone (i.e. the area in which the active agent of the delivery composition is to be released) within the body of the animal to which they are administered. Particularly, the compounds and compositions of the present invention are useful for orally administering active agents, especially those which are not ordinarily orally deliverable.

The present invention is particularly useful for the administration of polypeptides, including proteins, such as, but not limited to, therapeutical agents, nutritional products, mucopolysaccharides, lipids, carbohydrates, steroids, hormones, growth hormones (GH), growth hormone releasing hormones (GHRH), epithelial growth factors, vascular endothelial growth and permeability factors (VEGPF), nerve growth factors, cytokines, interleukins, interferons, GMCSF, hormone-like producst, neurological factors, neurotropic factors, neurotransmitters, neuromodulators, enzymes, antibodies, peptides, proteic fragments, vaccines, adjuvants, antigens, immune stimulating or inhibiting factors, heomatopoietic factors, anti-cancer products, anti-inflammatory agents, anti-parasitic compounds, anti-microbial agents, nucleic acid fragments, plasmid DNA vectors, cell proliferation inhibitors or activators, cell differentiating factors, blood coagulation factors, immunoglobulins, anti-angiogenic producst, negative selective markers or “suicide” agent, toxic compounds, anti-angiogenic agenst, polypeptides, and anti-cancer agent nucleotides, and the like, and structurally similar bioactive equivalents thereof.

In accordance with one embodiment of the present invention, there is provided a composition for oral administration and intestinal delivery of a nutritional compound or a therapeutic polypeptide that can be formulated, but without limitation to products described herein, with deoxycholate and saponins in a ratio to provide a substantially increased absorption and systemic bioavailability of the peptide by the intestine of the host. The composition also comprises a pH neutralizing agent, such as but not limited to sodium carbonate and calcium carbonate, and at least one inhibitor of digestive enzymes, such as but not limited to egg albumin or beans. A skilled artisan will understand that the nature, the number and the amount of neutralizing agents, digestive enzyme inhibitors and uptake increasing agents is adaptable to reach desired delivery properties. This composition is preferably solid so as to be easy to manipulate in formulating oral composition forms. Neutralization of pH is intended to mean increasing the pH into the digestive tract to acid-base equilibrium compatible with most of known active natural or synthetic biological products. The digestive tract's pH may be, but not limited to, between about 5 and 9, and preferably between about 6.5 and 7.5.

It is to be understood that the above list of drugs is for illustration purposes only and is not provided as an all inclusive list of all the drugs which may be beneficially formulated or reformulated using the oral delivery compositions of the present invention. Other physiologically-active compounds that can be encapsulated in the compositions of the present invention include biologically-active compounds, such as proteins, enzymes, anti-enzymes, peptides, catecholamines, anti-histamines, analgesics, and the like. For the purposes of the present invention “biological” is defined to mean any nutritionally or medically useful composition derived from a biological source and/or a synthetic pharmacological equivalent thereof such as insulin, heme, hemoglobin (bovine, human, or synthetic), and hormones; “enzyme” or “enzyme system” is defined to mean any protein or conjugated protein produced biologically or synthetically and which functions as a biocatalyst. Other medically useful compositions known to those skilled in the art, for example, globulin, one or more glycoproteins, such as erythropoeitin, may also be incorporated in the composition of the present invention.

The amount of therapeutic polypeptide will vary widely, depending on various factors such as the particular peptide to be delivered, the indication to be treated, the individual patient, and the like. The amount will be a therapeutically effective amount, that is, an amount that will provide a therapeutic effect, to be determined in accordance with well-established medical practice.

Another embodiment of the present invention is the use of enteric coatings, which are available for tablets and capsules. Enteric coatings will remain intact in the stomach but will rapidly dissolve once they arrive at the small intestine, thereafter releasing the drug at sites downstream in the intestine (e.g., the ileum and colon). Enteric coatings are well known in the art. Alternatively, a controlled release oral delivery vessel designed to release a drug after a predetermined period of time, and thus after the vessel has passed into the ileum or colon, can be used to deliver the formulation of the present invention. Such vessels include the CHRONSET™ delivery device (ALZA Corporation, Palo Alto, Calif.) and the Pulsincap™ delivery device (R.P. Scherer Co.).

The composition may further comprise an ion-pair forming reagent wherein the mole ratio of ion-pair forming reagent to drug is from about 2:1 to about 10:1. The ion-pair-forming reagent is added to increase the lipophilicity of the dissolved physiologically active agent or drug and thereby increase its membrane permeability. Increasing the drug's lipophilicity may also provide some protection of the drug from enzymatic deactivation as much of the peptide degradation that occurs in vivo does so in the aqueous environment of the gastrointestinal tract. Representative ion-pair forming reagents include sodium decanesulfonate, sodium lauryl sulfate, and sodium benzoate.

In one embodiment of the present invention, the composition may optionally comprise from about 0.01% to about 10% based on the total volume of the composition of an intestinal mucosal membrane transport enhancing agent, such as deoxycholate. Such agents facilitate the absorption of the therapeutic agent across the mucosal tissues in the intestinal mucosa and directly into the bloodstream of the subject. Also tissue transport enhancing agents suitable for use in the present compositions are selected from essential or volatile oils or from non-toxic, pharmaceutically acceptable organic and inorganic acids or salts and esters thereof. Essential or volatile oils which may be employed in the composition are selected from soybean oil, faba oil, rice oil, fish oil. The preferred essential oil is fish oil.

In another embodiment of the present invention, the composition may contain additional agents such as preservatives and antioxidants. Typical preservatives include but are not limited to sodium benzoate, sorbic acid, and the methyl and propyl esters of p-hydroxy-benzoic acid (parabens). Representative antioxidants include butylated hydroxy anisole, butylated hydroxy toluene, nordihydroguaiaretic acid, the gallates such as propyl gallate, hydroquinone, propenyl methyl guaethol and alkyl thiopropionates, or water soluble agents such as alkanolamines, alcohols, and propylene glycol. The most preferred antioxidant is Tenox™ GT1 (1:1 vitamin E-soybean oil), present in a concentration of between about 5% to about 25% based on the total volume of the droplet.

Oral absorption of recombinant human GH to carp is enhanced up to a 1000-fold when the delivered together with deoxycholate.

To prepare the pharmaceutical formulation of the present invention, the ingredients are dry blended together, after which a small amount of oil is added. These materials are mixed together until a homogeneous mixture of ingredient results. The resulting solid formulation can be pressed into tablets that can then be coated with a suitable enteric coating. Alternatively, the solid formulation can be placed into a capsule formed of gelatin or the like and coated with an enteric compound, or placed into a controlled release delivery device such as the CHRONSET™. The solid formulation provides a mean for easily and conveniently fabricating a dosage form.

One embodiment of the invention is to provide a method for delivering hormones and pharmaceuticals to an animal or human host. Among the agricultural production field, the production of different species of fishes is importantly pointing out. The control of the reproduction physiology is of particular importance. The first indication of the invention for manipulating aquatic animals reproduction by feeding bioactive materials was provided by studies which employed mammalian and amphibian pituitaries. Thus, dietary replacement or supplementation with pituitary preparations has been observed to induce nuptial coloration in the bitterling Acheilognathus inter-medium, partially mature, and increase egg diameter, in the loach Misgurnus anguillicaudatus, resulting in ovulation and a shortening in brood interval by 10-15 days in the swordtail Xiphophorus helleri, and increase egg size and induce precocious maturation in female lake trout Salvelinus fontinalis. Similarly, oral administration of salmon pituitary extract to goldfish C. auratus, induced ovulation and increased spermiation. The importance of these data relates to the accompanying elevations in plasma salmon gonadotropin (sGtH), testosterone and 17α-20β-diphydroxy-4-pregnan-3-one, which provide a likely endocrine-based explanation for the observed effects of other pituitary preparations during earlier investigations (i.e., uptake of GtH).

Due to the problems inherent to the use of pituitary preparations and partially purified hormones, it would appear unlikely that such preparations will offer any major benefit with respect to the control of reproduction in cultured species using the oral route unless formulated in a composition of the present invention. A comparatively recent innovation in the control of maturation has been the application of gonadotropin-releasing hormone. Many of the analogue forms of GnRH are 50-100 times more effective at inducing ovulation than the natural forms, and include those which incorporate D amino acids and have terminal residues substituted with ethylamide. These manipulations have the effect of enhancing the resistance of the molecule to proteolysis. GnRHs stimulate the natural release of GtH, exhibit wide species potency, are relatively easy to manufacture and therefore, are economical. In addition, the peptides are stable over a wide range of temperature, and express non-varying potency. Importantly, the peptides are stable for an indefinite period provided they are stored in sterile conditions at temperatures below −20° C. As such GnRHAs provide excellent candidate molecules for use in the oral approach to controlling reproduction. Indeed, sufficient experimental evidence has accumulated, such that dietary delivery of GnRHAs, with or without dopamine agonist is now indicated as a method for controlling the final stages of maturation in fish. While more expensive than traditional methods (injection, implantation), dietary administration offers the advantage of being stress-free. This advance in reproductive biotechnology is particularly useful for species which are vulnerable to handling and/or, are too small for safe injection (i.e., ornamental species). In addition, chronic treatment with GnRHAs provides means to induce maturation precociously, which is considered advantageous during roe production, or for use with sex reversed broodstock.

Similar to the control of reproduction, studies using pituitaries as feed supplements also provide an early indication of the possibility of manipulating growth in teleosts using oral delivery techniques. Thus, it has been observed that the feeding of gruppies Lebistes reticulatus, with anterior pituitary powder resulted in significantly enhanced growth performance when compared against controls. Also, a 50% increase in length was observed for swordtails fed exclusively on dried anterior pituitary from birth, while other experiments observed a doubling in weight and tripling in length of lake trout fed anterior pituitary twice weekly. Treatment of cultured teleosts with growth hormone (GH) and related peptides offers a number of potential advantages, and several studies have confirmed that orally delivered GH not only enters the bloodstream, but accelerates growth rate in fish. Supplies of recombinant GH are presently stable and production could be increased many-fold with increased demand. Moreover, such recombinant proteins, when produced at the industrial level, are cost efficient and easily incorporated into commercial diets. While the structural integrity of the GH molecule may be of importance as a precursor to post-translational modified forms, methods of enhancing the molecules structural integrity or potency may provide benefit from an oral administration perspective. Description of growth-promoting fragments of the GH molecule may also provide products that express greater stability under lumenal degradation.

A particular embodiment of the present invention is to provide a composition and a method allowing the use of the oral route for vaccination that offers significant advantage in that it reduces labor costs, is time-saving, decreases the possibilities for cross-contamination with needles and does not involve inventory handling or require disposal of treatment waters.

The principal determinant of specific immunity at mucosal surfaces is secretory IgA (S-IgA) which is physiologically and functionally separate from the components of the circulatory immune system. S-IgA antibody responses may be induced locally by the application of suitable immunogens to a particular mucosal site. The bulk of mucosal S-IgA responses, however, are the results of immunity generated via the common mucosal immune system (CMIS), in which immunogens are taken up by specialized lympho-epithelial structures, collectively referred to as mucosa-associated lymphoid tissues (MALT). The best immunologic lymphoepithelial structures are the gut-associated lymphoid tissues (GALT), such as intestinal Peyer's patches. Other structurally and functionally similar lymphoid follicles occur at other mucosal surfaces, including those of the respiratory tract.

According to the present invention, a host can be immunized by oral administration of bacterial protein immunogens, preferably mixed with an adjuvant, such as cholera toxin (CT). Of course, as an adjuvant, the amount of cholera toxin used is non-toxic to the host.

The ability of a vaccine to protect against microbial colonization, as provided herein, means that the active component may protect against disease not only in the immunized host but, by eliminating carriage among immunized individuals, the pathogen and hence any disease it causes may be eliminated from the population as a whole.

Oral administration may also prevent sepsis resulting from administration of microbials, so that the vaccine can protect against both microbial colonization and sepsis (systemic infection).

For example, PspA is a preferred antigen for pneumococal infections. In published International patent application WO 92/14488, the entire content of which is incorporated herein by reference there are described DNA sequences for the PspA gene from S. pneumoniae Rx1, the production of a truncated form of PspA by genetic engineering and the demonstration that such truncated form of PspA confers protection in mice to challenge with live pneumococci.

From sequences of the PspA gene, it has been shown that PspA proteins are variable in size (roughly 70 kDa). The C-terminal 37% of the molecule is largely composed of the 20-amino acid repeats which form a binding site that permits PspA to attach to the phosphocholine residues of the pneumococcal lipoteichoic acids. The central region of PspA is rich in prolines and is suspected to be the portion of the molecule that passes through the cell wall. The sequence of the N-terminal 80% of the molecule is largely beta-helical and contains the region of PspA that can elicit antibodies that are protective against sepsis. Although PspA's are almost always at least slightly different from one another, there is enough cross-reactivity between them that antibodies or an immunological response to one PspA detect or is effective with respect to PspAs on all pneumococci. Moreover, immunization with one PspA can either protect against death or delay death with virtually all-different challenge strains. Accordingly, a mixture of a small number of PspA's could provide effective immunity against most pneumococci.

The immunoprotective truncated PspAs described in WO 92/14488 may be used in the present invention as the PspA fragments described above for oral administration.

Different vector systems for in vitro and in vivo expression of recombinant proteins are known; e.g., bacterial systems such as E. coli; and virus systems such as bacterial viruses, poxvirus (vaccinia, avipox virus, e.g., canarypox virus, fowlpox virus), baculovirus, herpes virus; yeast; and the like; and, these systems may be used for producing recombinant PspA using the coding gene thereof.

Immunogenicity may be improved if the antigen is co-administered with an adjuvant, commonly used as 0.001% to 50% percent solution in phosphate buffered saline. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants may also attract cells of the immune system to an antigen depot and stimulate such cells to elicit immune responses.

Immunostimulatory agents or adjuvants have been used for many years to improve the host immune response to, for example, vaccines. Intrinsic adjuvants, such as lipopolysaccharides, normally are the components of the killed, attenuated or non-toxic bacteria used as vaccines. Extrinsic adjuvants are immunomodulators which are typically non-covalently linked to antigens and are formulated to enhance the host immune response. Aluminum hydroxide and aluminum phosphates (collectively commonly referred to as alum) are routinely used as adjuvants in human and veterinary vaccines. The efficacy of alum in increasing antibody responses to diphtheria and tetanus toxoids is well established and, more recently, a HBsAg vaccine has been adjuvanted with alum.

A wide range of extrinsic adjuvants can provoke potent immune responses to antigens. These include saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), toxins as well as lipid A, liposomes and nucleic acids. To efficiently induce humoral immune response (HIR) and cell-mediated immunity (CMI), immunogens are preferably emulsified in adjuvants.

Compositions of the invention, especially for oral administration may be conveniently provided as liquid preparations, e.g., isotonic aqueous solutions, suspensions, emulsions or viscous compositions which may be buffered to a selected pH. However, since delivery to the digestive tract is preferred, compositions of the invention may be in a “solid” form of pills, tablets, capsules, caplets and the like, including “solid” preparations which are time-released or which have a liquid filling, e.g., gelatin covered liquid, whereby the gelatin is dissolved in the stomach and/or small intestine for delivery to the gut and/or digestive system.

The composition of the invention may also contain pharmaceutically acceptable flavoring and/or coloring agents for rendering them more appealing. The viscous compositions may be in the form of gels, lotions, ointments, creams and the like and will typically contain a sufficient amount of a thickening agent so that the viscosity is from about 2500 to 6500 cps, although more viscous compositions, even up to 10,000 cps may be employed. Viscous compositions have a viscosity preferably of 2500 to 5000 cps, since above that range they become more difficult to administer. However, above that range, the compositions can approach solid or gelatin forms that are then easily administered as a swallowed pill for oral ingestion.

Liquid preparations are normally easier to prepare than gels and other viscous compositions, and solid compositions. Additionally, liquid compositions are somewhat more convenient to administer, especially to animals, children, particularly small children, and others who may have difficulty swallowing a pill, tablet, capsule or the like, or in multi-dose situations. Viscous compositions, on the other hand can be formulated within the appropriate viscosity range to provide longer contact periods with mucosa, such as the lining of the stomach or intestine.

Suitable nontoxic pharmaceutically acceptable carriers, and especially oral carriers, will be apparent to those skilled in the art of pharmaceutical and especially oral or peroral pharmaceutical formations. Obviously, the choice of suitable carriers will depend on the exact nature of the particular dosage form, e.g., liquid dosage form (e.g., whether the composition is to be formulated into a solution, a suspension, a gel or another liquid form, or a solid dosage form, or e.g., whether the composition is to be formulated into a pill, tablet, capsule, caplet, time release form or liquid-filled form).

Solutions, suspensions and gels, normally contain a major amount of water (preferably purified water) in addition to the antigen. Minor amounts of other ingredients such as pH adjusters (e.g., a base such as NaOH), emulsifiers or dispersing agents, buffering agents, preservatives, wetting agents, jelling agents, (e.g., methylcellulose), coloring and/or flavoring agents may also be present. The compositions can be isotonic, i.e., it can have the same osmotic pressure as blood and lacrimal fluid.

The desired isotonicity of the composition of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Viscous compositions are normally prepared from solutions by the addition of such thickening agents.

A pharmaceutically acceptable preservative may be employed to increase the shelf life of the composition. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the composition must be selected to be chemically inert with respect to microbial antigens. This will present no problem to those skilled in chemical and pharmaceutical principles, or problems can be readily avoided by reference to standard tests or by simple experiments (not involving undue experimentation), from this disclosure.

The immunologically effective compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally the pH may be from about 3 to 7.5. Compositions can be administered in dosages and by techniques well known to those skilled in the medical and veterinary arts taking into consideration such factors as the age, sex, weight, and condition of the particular patient or animal, and the composition form used for administration (e.g., solid vs. liquid). Dosages for humans or other mammals can be determined without undue experimentation by the skilled artisan.

When CT is used as an adjuvant for oral immunizations, specific IgA antibodies are induced in secretions. Strong circulatory immune responses can also be induced, with IgG and IgA antibodies in the serum, and IgG and IgA antibody-secreting cells in the spleen. The circulatory (or systemic) immune responses elicited by oral (peroral; intragastric) administration of microbial antigens along with CT are comparable with, or even stronger than, those induced by the administration of similar immunogens by the intragastric route. Accordingly, it appears that oral immunization is an effective route for stimulating common mucosal responses as well as circulatory antibody responses and can require less antigen than other immunization routes.

Most soluble or non-replicating antigens are poor immunogens, especially by the peroral route, probably because they are degraded by digestive enzymes and have little or no tropism for the GALT. A notable exception is CT, which is a potent mucosal immunogen, probably because of the G.sub.M1 ganglioside-binding property of this binding subunit, CTB, that enables it to be taken up by the M cells of Peyer's patches and passed to the underlying immunocompetent cells. In addition to being a good mucosal immunogen, CT is a powerful adjuvant. When administered in micrograms doses, CT greatly enhances immunogenicity of other soluble antigens co-administered with it.

In one embodiment, and in accordance with the present invention, there is provided a process for treating a disease or disorder of a host by delivery of a therapeutic agent to the host after oral administration.

In another embodiment, cancer cells that may be treated in accordance with the present invention include malignant tumors. Malignant (including primary and metastatic) tumors which may be treated include, but are not limited to, those occurring in the adrenal glands; bladder, bone; breast; cervix; endocrine glands (including thyroid gland, the pituitary gland, and the pancreas); colon; rectum; heart; hematopoietic tissue; kidney; liver; lung; muscle; nervous system; brain; eye; oral cavity; pharynx; larynx; ovaries; prostate; skin (including melanoma); testicles; thymus, and uterus. It is to be understood, however, that the scope of the present invention is not to be limited to the treatment of any particular tumor.

It is to be understood, however, that the scope of the present invention is not to be limited to specific biologically active ingredients, as therapeutic agents.

In accordance with another preferred embodiment of the present invention, the agent which is capable of inhibiting, preventing, or destructing the cancer cells upon delivery of such agent is a negative selectable marker; i.e. a material which in combination with a chemotherapeutic or interaction agent inhibits, prevents or destroys the growth of the cancer tumor cells.

Thus, upon systemic delivery of negative selective marker, an interaction agent is administered to the animal or human host. The interaction agent interacts with the negative selective marker in order to prevent, inhibit, or destroy the growth of the cancer Negative selective markers which may be employed for example, but are not limited to, thymidine kinase, and cytosine deaminase.

The interaction agent is administered in an amount effective to inhibit, prevent, or destroy the growth of the cancer cells. For example, the interaction agent may be administered in an amount from about 5 mg to about 15 mg/kg of body weight, preferably about 10 mg/kg, depending on overall toxicity to a patient.

The present invention will be more readily understood by referring to the following examples which are given to illustrate the invention rather than to limit its scope.

### EXAMPLE I

### Growth Enhancement of Rainbow Trout (Oncorhynchus mykiss) and Brook Trout (Salvelinus fontinalis)

The aquaculture industry worldwide has undergone rapid expansion during the past 2 decades and currently represents the fastest growing agricultural segment. The sector has grown at an annual percentage rate of 10.9 since 1984, compared with 3.1 for terrestrial livestock meat production. The fastest growing livestock sector over the same period was chicken meat production with an APR of 5.3, followed by pig meat 3.4, mutton and lamb 1.4, and beef and veal 0.9. Aquaculture's contribution toward total world food fish landings has increased more than two fold since 1984 from 11.5% to 25.6% by weight in 1995. Projected increased demand for seafood products, coupled with decreased fisheries landings from wild stocks has, and will continue to contribute to the growth of the aquaculture industry.

The aquaculture industry, like other sectors of agriculture, faces many of the production challenges associated with traditional livestock production. Forty to fifty percent of the cost of salmonid production is attributed to feeding. Rations contain a high percentage of costly fish protein and salmonids require a relatively long feeding period to reach market weight. In fast growing fish, excessive fat deposition is a concern to both producers and consumers.

The goal of the food animal industry is to optimize production efficiency by minimizing the input of feed, labour, and capital investment while maximizing the yield of high quality protein. In the past, economically important parameters have been altered by genetic selection or nutritional modification. More recently, a variety of approaches have emerged involving endocrine system manipulation to influence growth and body composition of domestic animals. The ability of exogenous compounds to successfully alter the growth performance of domestic animals and offer potential savings in production costs, has prompted investigations into the use of these agents in fish.

The administration of growth hormone (GH) derived from various sources has provided evidence that this hormone plays a key role in stimulating somatic growth and reducing fat deposition in fish. Both native and recombinant piscine GH has been applied to several fish species, and are equipotents when injected into intact salmonids. As well, GH derived from mammalian and avian sources have been reported to be effective in altering growth performance of juvenile salmonids. Administration of bovine GH (bGH) to salmonids leads to a two to three-fold increase in growth rate, an increase in appetite and feed efficiency and reduction in adipose tissue. Exogenous GH is also effective in older (subadult) fish, and at low water temperatures when growth rate is depressed.

Oral application of GH is a practical method it has provided histochemical and biological evidence for a mechanism which transports intact proteins into the circulation of teleost fish following oral administration. It is now shown that orally administered horseradish peroxidase is transported to the circulation within 1 h.

It is reported the transfer of bGH into the circulatory system of yearling rainbow trout following introduction of the hormone into the lumen of the digestive tract. Similarly, it is demonstrated that recombinant salmon growth hormone (rsGH) administered orally, significantly elevated plasma rsGH concentrations. This same results show that weekly intragastric administration of rsGH resulted in a 50% increase in weight gain and fish length compared to control fish.

The above research supports that orally administered GH from various sources may influence growth performance in several teleost fish species, by being protected to eliminate gastric and intestinal digestion so that it remains intact and biologically active. This has given rise to several attempts to develop systems that will protect bioactive proteins (growth hormones, antigens, etc.) from the acidic environment of the stomach. Oral or rectal intubation of fish are effective methods to deliver bioactive proteins past the stomach, however, they are not feasible for commercial application. Attempts have been made to co-administer bioactive proteins along with detergents and antacids to reduce the acidic environment in the stomach. While these studies have demonstrated reduced protein degradation, the treatments used may affect the uptake of other important dietary factors. Another option is the use of pH-sensitive polymers which encapsulate and protect the peptides from acidic degradation in the stomach and permit release once in the small intestine.

It is clearly shown from the method of the present invention that compounds (hormones, vaccines, antibodies etc) are delivered orally past the stomach of monogastic animals (including humans) in order to bypass gastric digestion to the site of absorption in the small and/or large intestine(s). To date, a large proportion of this work has centered on developing encapsulation strategies using a range of formulations and forms of interest. These formulations and forms may simply modulate the release of a specific compound in a predetermined fashion, or may use specific physiological determinants (e.g. pH, temperature etc.) to trigger the delivery of the encapsulated material.

Complex polymer used in other forms of delivery systems is sometimes difficult to characterize. As well, the utilization of certain polymers that are not Generally Regarded as Safe (GRAS) makes regulatory approval of these systems a long and risky process. Furthermore, many polymer systems are relatively costly making large-scale utilization impractical.

The current experiment highlights a new strategy to permit oral delivery of a bioactive peptide (in this case bST). By feeding a bioactive compound of interest along with a cocktail of antinutritional factors to temporarily suppress digestive enzyme function and products that augment intestinal absorption (referred to as the ‘bypass cocktail’), we have shown that we can effectively bypass the enzymatic process, and enhance intestinal uptake of the aforementioned compound to achieve a desired biological effect.

Materials and Methods

Bypass Cocktail Formulation

The formulation of the bypass cocktail is shown in Table 1. Unprocessed oilseed and pulse ingredients were obtained from local suppliers and mechanically dehulled. Fish meal, rice bran, brewers yeast, sodium carbonate, calcium carbonate and EDTA were all feed grade and purchased from local suppliers. Sodium deoxycholate and crude egg albumin were purchased from Sigma Chemical Co. (St Louis Mo.). The diet was mixed as indicated and ground using a 1 mm mesh.

TABLE 1
Bypass cocktail formulation

| Ingredient | Inclusion Rate (g/kg) |
|---|---|
| Fish meal | 150 |
| Egg albumin | 100 |
| Beans | 300 |
| Sodium carbonate | 100 |
| Calcium carbonate | 100 |
| Fish oil | 100 |
| Rice bran | 50 |
| EDTA | 50 |
| Brewers yeast | 45 |
| Sodium deoxycholate | 4-40¹ |

¹Concentration varied in experiments see Materials and Methods section

Fish and Feeding

A series of experiments using two salmonid species was undertaken (Experiment 1: rainbow trout; Experiment 2: brook trout). These species were chosen as they represent both well studied experimental models as well as economically important cultured species.

For Experiment 1, rainbow trout (n=20; initial weight=52 grams) were stocked into 6-60 liter cylindro-conic tanks in a closed water recirculation system 2 weeks prior to the start of the experiment. Water temperature was held at 15° C. and photoperiod was set at 12hL:12hD cycle. In Experiment 2, brook trout (n=400; initial weight 38 grams) were stocked into 8-800 liter cylindro-conic tanks in a closed water recirculation system two weeks prior to the start of the experiment. Water temperature was 11° C. for the duration of the experiment; fish were subjected to natural photoperiod (approx. 14hL:10hD). During both experiments water quality (ammonia, nitrite) was monitored weekly and oxygen concentrations measured daily. Fish were fed a commercial feed (Corey Feed Mills Ltd. Fredericton, NB) during the acclimation period and during the non-treatment periods.

Experimental Manipulations

In Experiment 1, recombinant bovine somatotropin (rbST; Monsanto Co. St Louis Mo.) was included to provide fish with 20 μg/g fish. Three duplicate groups were fed varying levels of provide either 0 (control), 4 or 40 g sodium deoxycholate/kg bypass cocktail. In the second experiment, 4 duplicate treatment groups received food supplemented with 0, 1, 5 or 10 mg deoxycholate/kg bypass cocktail with 20 μg rbST/g fish.

In both Experiments 1 and 2, fish weight and feed consumption were monitored on a weekly basis. Fish were weighed and then fasted for 36 hours prior to feeding the bypass cocktail containing bST. Following feeding feed was withheld for an additional 12 hours. From this point, fish were fed twice daily to near satiation.

Results

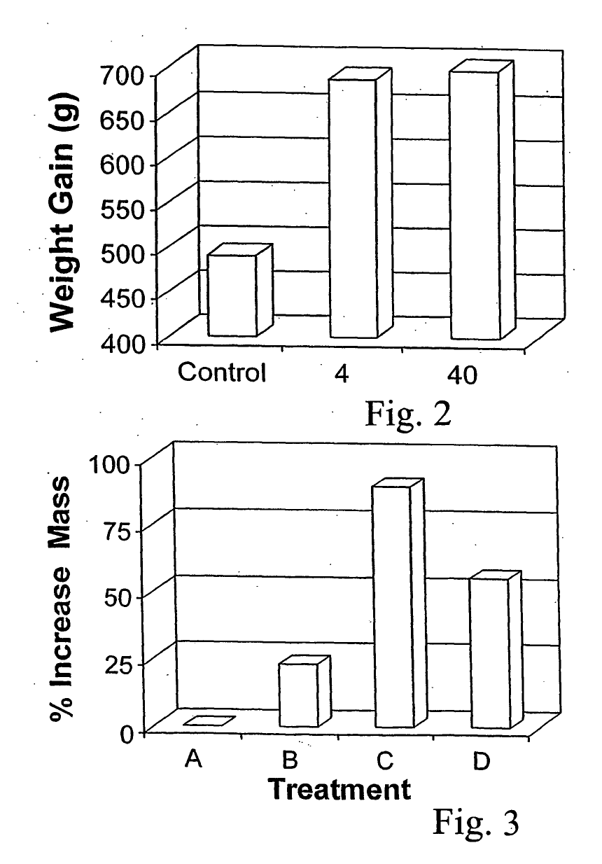

In both experiments, no treatment-associated mortalities were noted, suggesting no adverse health effects of the bST or the bypass cocktail on rainbow and brook trout. Fish fed bST in the bypass cocktail had significantly improved growth rates versus controls. In Experiment 1, treated fish averaged a 25% increase in growth rates; the fastest growing tanks grew over 40% larger than controls (FIGS. 1 and 2). In Experiment 2, bST-treated groups showed improved growth rates versus controls, though those fed the bypass cocktail containing 5 g/kg deoxycholate showed the highest growth rates with a 90% increase in growth rates over controls (FIG. 3).

### EXAMPLE II

### Growth Enhancement of Rainbow Trout (Oncorhynchus mykiss)

Method

Intraperitoneal (IP) administration dose per fish weekly was 20 μg bST/g live body weight for 6 weeks.

Results

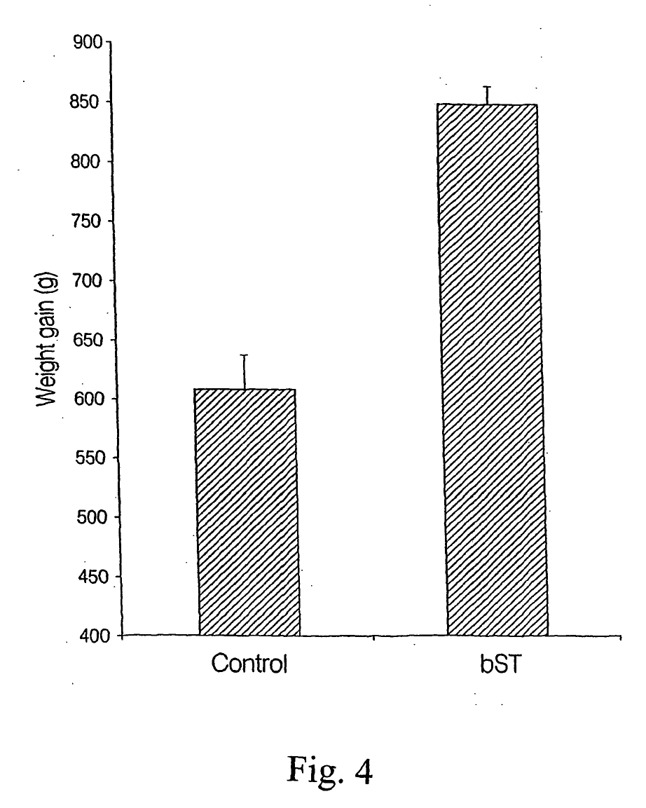

FIG. 4 illustrates that recombinant bST injected IP significantly induces increased body weight gain in rainbow trout.

### EXAMPLE III

### Assessment of Proteolytic Enzyme Inhibitors Present in Feed Ingredients

Extract Enzymes Protocol

Materials

1. Centrifuge Sorvall model
2. Bench-top blender
3. Dissecting material (scissors)
4. Centrifuge bottle
5. Disposable cuvettes for spectrophotometer
6. Microcentrifuge tubes 1.5 ml
7. Spectrophotometer
8. Vortexer
9. Microplates reader from Biorad
10. 50 mM Tris-HCl pH=7.5
11. Commassie blue staining solution
12 BSA (1 mg\ml) standard solution
13 TCA 20%
14. Rainbow trouts pancreatic and duodenal tissues
15.0.5% casein in 50 mM Tris-HCl pH=9
16.50 mM Tris-HCl+CaCl₂ 10 mM pH=7.5
   Enzyme Extract
1. Rainbow trout were weighed and sacrificed.
2. Dissection was performed to remove the proximal small intestine from the fish.
3. After weighing, the tissues were homogenised in 50 mM Tris-HCl ph=7.5 (1:10 w\v).
4. Centrifuge at 16000×g for 30 min at 4° C.
5. Keep the supernatant. Aliquot and store them at −20 C for further use.
6. Perform Commassie assay to measure the amount of protein present in the enzyme extract.
   Commassie Blue Stain Protocol
1. Weigh 160 mg of BSA in 10 ml of 50 ml Tris-HCl pH=7.5.
2. Prepare a standard curve of BSA (0 μg/ml to 1600 μg/ml).
3. Add 4 μl in each well of either BSA, extract enzyme and dilute (1:1) extract enzyme in a 96 well plate.
4. Add 200 μl of Commassie blue.
5. Read at 655 nm with the microplate reader from Biorad.
   Enzymatic Protocol:
   The experiments are performed in duplicate.
   BLANK:
1. To 500 μl of 50 mM Tris-HCl+10 mM CaCl₂ pH=7.5 solution.
2. Add 500 μl of TCA 20% (distilled water) solution.
3. Add 20 μl of enzyme extract
4. Add 500 μl of casein 0.5% (50 mM Tris-HCl pH=9) solution.
5. Incubate 15 min at 4° C. (ice). Centrifuge at 12000×g for 5 min and read at 280 nm.
   TEST:
1. To 500 μl of 50 mM Tris-HCl+10 mM CaCl₂ pH=7.5 solution.
2. Add 20 μl enzyme extract.
3. Add 500 μl of casein 0.5% (50 mM Tris-HCl pH=9) solution.
4. Incubate at 0, 5, 10, 15 and 30 min at room temperature.
5. Stop the reaction by adding 500 μl de TCA 20%. Incubate 15 min at 4° C. (ice). Centrifuge at 12000×g for 5 min and read at 280 nm.
   Inhibitor Extraction
1. Grind with the industrial grinder the food bought commercially in fine powder.
2. Weigh 250 mg of the powder and put it in 10 ml of 50 mM Tris-HCl pH=7.5 (final concentration should be 25 mg/ml).
3. With the hand tissues grinder, homogenise the solution.
4. Centrifuge 2000×g for 10 min at room temperature*.
5. Keep the supernatant. It will be the inhibitor extract for the enzymatic protocol.
   Enzymatic Protocol:
   The experiments are performed in duplicate
   BLANK:
1. To 500 μl of 50 mM Tris-HCl+10 mM CaCl₂ pH=7.5 solution.
2. Add 500 μl of TCA 20% (distilled water) solution.
3. Add variable volumes of inhibitor extract or 50 mM Tris-HCl pH=7.5 solution.
4. Add 10 μl of enzyme extract.
5. Add 500 μl of casein 0,5% (50 mM Tris-HCl pH=9) solution.
6. Incubate 15 min at 4° C. (ice). Centrifuge at 12000×g for 5 min and read at 280 nm.
   CONTROL:
1. To 500 μl of 50 mM Tris-HCl+10 mM CaCl₂ pH=7.5 solution.
2. Add variables volumes of 50 mM Tris-HCl pH=7.5 solution.
3. Add 10 μl enzyme extract.
4. Incubate 60 min at room temperature.
5. Add 500 μl of casein 0.5% (50 mM Tris-HCl pH=9) solution.
6. Incubate at 30 min at room temperature.
7. Stop the reaction by adding 500 μl de TCA 20%. Incubate 15 min at 4° C. (ice). Centrifuge at 12000×g for 5 min and read at 280 nm.
   TEST:
1. To 500 μl of 50 mM Tris-HCl+10 mM CaCl₂ pH=7.5 solution.
2. Add variables volumes of inhibitor extract.
3. Add 10 μl enzyme extract.
4. Incubate 60 min at room temperature.
5. Add 500 μl of casein 0.5% (50 mM Tris-HCl pH=9) solution.
6. Incubate at 30 min at room temperature.
7. Stop the reaction by adding 500 μl de TCA 20%. Incubate 15 min at 4° C. (ice).
8. Centrifuge at 12000×g for 5 min and read at 280 nm.
   Results

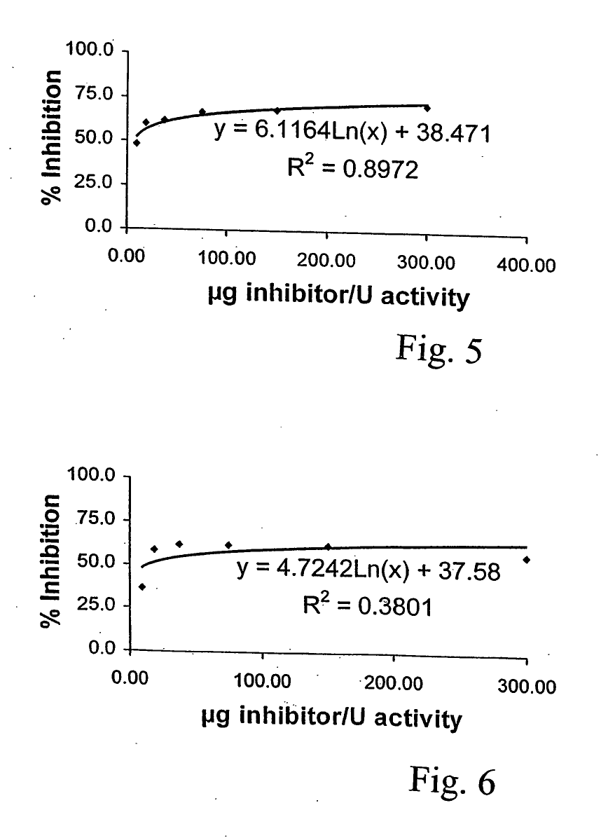

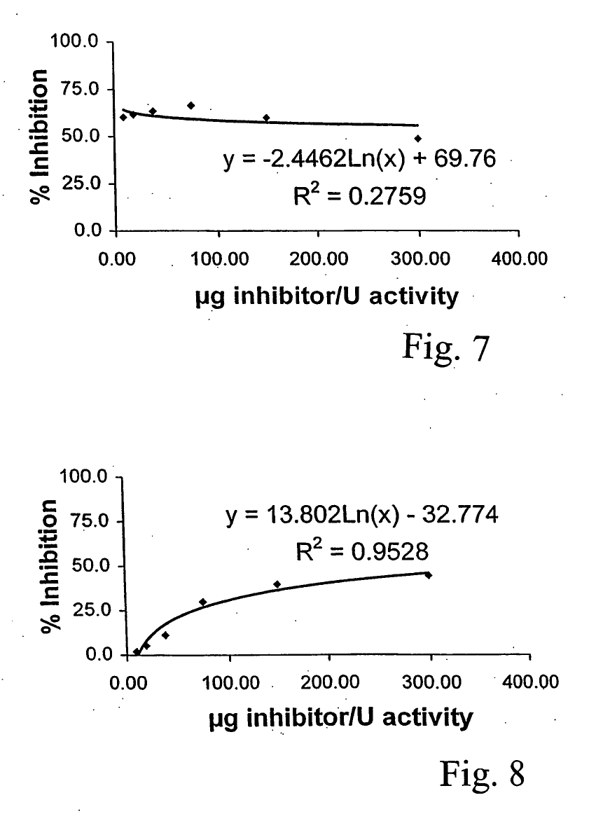

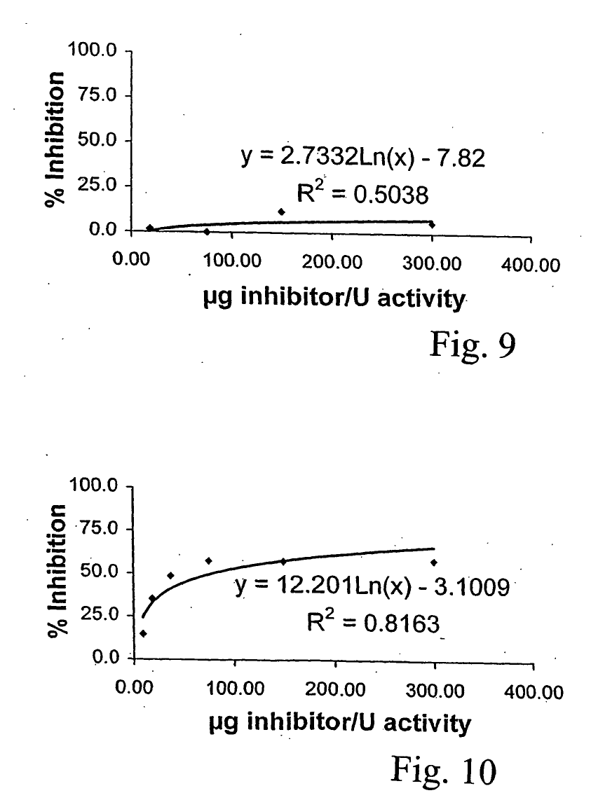

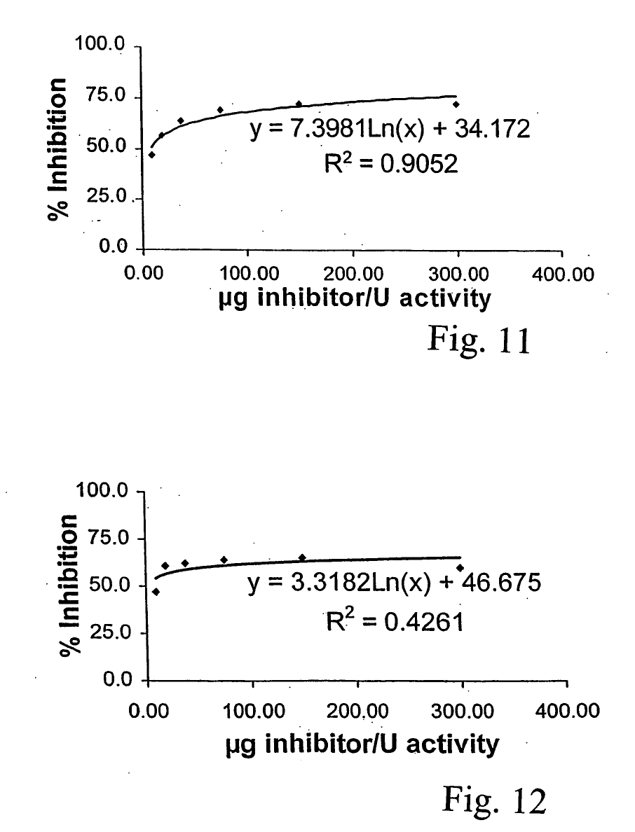

FIGS. 5-12 demonstrate the effects of individual protease inhibitor components of the Oralject™ cocktail on in vitro proteolytic inhibition, as well as the overall inhibition of the Oralject™ cocktail. These data are presented as the degree of proteolytic enzyme inhibition versus increasing level of inhibitor inclusion. The data reveal that the individual components (lyophylized ovalbumin, red kidney beans, soybeans, faba beans, EDTA, wheat bran, spray-dried ovalbumin, FIGS. 5-12 respectively) of the Oralject™ cocktail affect to differing degrees the inhibition of in vitro proteolytic enzyme activity. Furthermore the overall cocktail is effective in inducing-overall proteolytic enzyme inhibition. Finally, using the curves generated in FIGS. 5 to 12, the point of maximal inhibition as well as the concentration of inhibitor providing 50% of the maximal inhibition were extrapolated.

### EXAMPLE IV

### Enzyme Assay for the Quantification of Horseradish Peroxidase in the Blood of Rainbow Trout

Material

1. 96 well plates (Immulon™ II from VWR)
2. Microplate reader from Biorad T
3. Microcentrifuge tubes of 1.5 ml
4. Centrifuge tubes of 15 ml or 50 ml
5. TMB tablets
6. Horseradish peroxidase type 1 (Sigma)
7. Anti-Horseradish peroxidase from goat IgG (ICN)
8. 0,1M carbonate-bicarbonate pH=9.6 buffer
9. 0,1 M phosphate-citrate pH=5 buffer
10. PBS 1×+BSA 1%+0,5% Tween 20 buffer
11. PBS 1×pH=7,4 buffer
12. Hydrogen peroxide 30%
13. Saran wrap
14. Incubator at 37° C.
15. Distilled water
16. Rainbow trout (plasma)
   Method
   Coat Plate with Antigen
1. Dispensed 200 μl of the anti-HRP from goat IgG dilute 1:1000 solution (in 0.1M carbonate-bicarbonate pH=9.6 buffer) into each well of a 96 wells plate.
2. Wrap coated plate in saran™ wrap to seal and incubate overnight at 4° C. or 2 hours at 37° C.
3. Rinse coated plate 3 times with PBS 1×pH=7.4. Each time, flick the phosphate-buffered saline into the sink and rinse 3 more times with distilled water.
4. The plates were shaken dry and stored at 4° C. until use.
   Block Residual Binding Capacity of Plate
1. Fill each well with 200 μl of PBS 1×+BSA 1%+0.5% Tween 20 buffer.
2. Incubate 30 minutes at room temperature.
3. Add 100 μl of the sample containing HRP dilute 1:10 in some wells.
4. Add 100 μl of standard curve plasma in the others wells.
5. Wrap the plate in the saran wrap and incubate 1 hour at 37° C.
6. Rinse 3 times with PBS 1×+BSA 1%+0.5% Tween 20 buffer.
   Standard Curve Method
1. Dilute the plasma of the rainbow trout 1:10 with PBS 1×pH=7.4.
2. Add HRP to obtain a final concentration of 0.5 to 8 ng/ml.
   Enzyme Assay
1. Add 200 μl of TMB (in 50 mM citrate-phosphate pH=5 buffer+30% of hydrogen peroxide) in each well.
2. Wait 30 minutes and add 50 μl of 1 M sulfuric acid to fix the coloration.
3. Read at 415 nm with the Microplate reader from Biorad™.
   Results

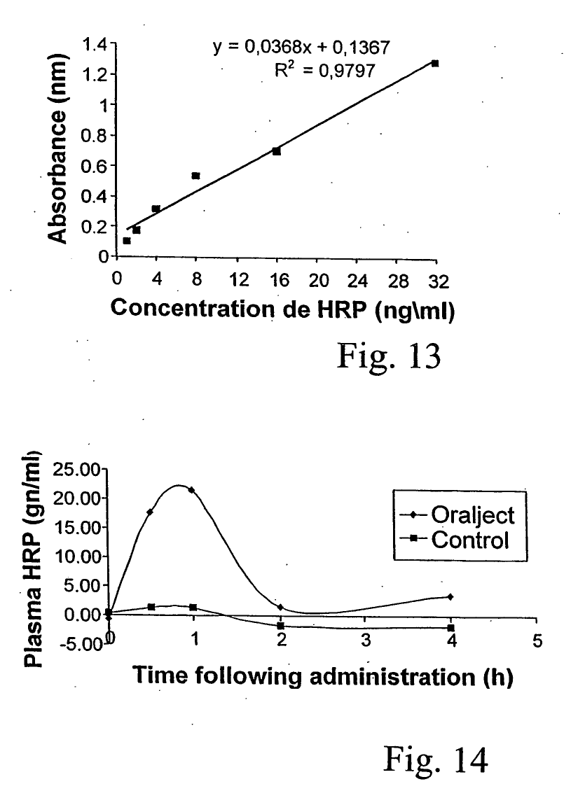

An ELISA was developed for horseradish peroxidase (HRP), permitting its use as a tracer for plasma uptake studies following oral administration. This method has provided an extremely sensitive method to document HRP uptake with lower detection limit of approximately 2.5 ng HRP/ml plasma and a linear portion up to 8 ng/ml (FIG. 13).

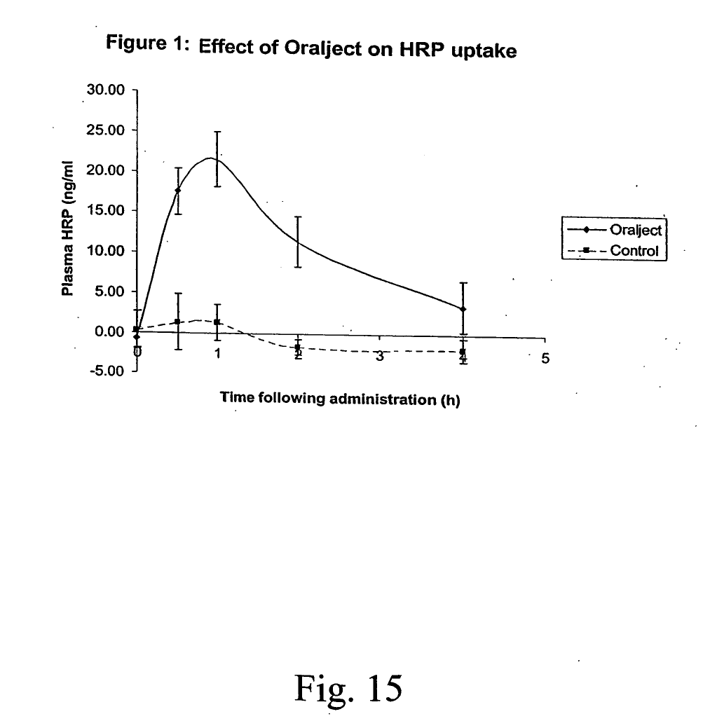

Using this method to follow HRP uptake, a fish meal-based control matrix, and the Oralject™ cocktail containing HRP (2.5 ng/g) was force fed to rainbow trout and blood samples taken at selected times following administration. As illustrated in FIG. 14, plasma uptake of orally delivered HRP in the Composition of the present invention was significantly higher than that of the fish meal control. Furthermore, the circulating concentrations of HRP were detected for a period of 6 h following administration.

### EXAMPLE V

### Oral Vaccine Delivery

Materials and Methods

For each vaccine, rainbow trout (n=50; housed in recirculated water system at 12° C.) were divided into 5 groups. Two groups of fish received the vaccine in the Oralject Formulation 1 (A. salmonicida: 40 mg/fish) either once bi-weekly for 6 weeks, or on three consecutive days, bi-weekly for 6 weeks. Treatment was similar for the Oralject Formulation 2. An identical protocol was used for the V. salmonicida vaccine, with the exception that fish were fed orally at 10 mg/fish. For each vaccine, an additional group included injected controls (200 ul/fish of 0.6×10 10 cells/ml for A. salmonicida and V. salmonicida) Blood samples were taken at 2, 4 and 6 weeks following the start of the experiment and serum collected and stored at −80° C. until required for analysis. A schematic of the experimental design is as follows: 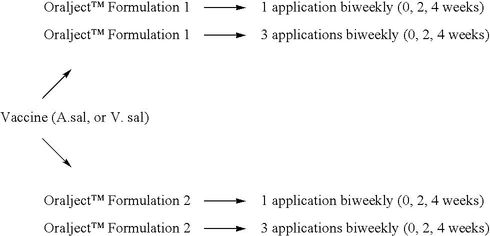
Elisa Assays

Antibody levels were determined using an ELISA protocol provided by ALPharma™. This method employed a mouse anti-salmon IG monoclonal antibody (4C10) and a peroxidase-labelled rabbit anti-mouse secondary antibody diluted appropriately. Fish serum samples were serially diluted 1:2 (6-fold; initial dilution: 1:10), although for comparison purposes, the resulting optical density from fish vaccinated orally was expressed as a percentage OD from a pool of fish serum (both diluted 1:100) collected from selected high titre, injected control fish group 6.

Results

Oral Application of HRP

FIG. 15 reveals that intact HRP was absorbed into the general circulation of fish within 30 minutes post-treatment. HRP concentration was maximized at 1 h and declined thereafter. There was negligible uptake of HRP in fish meal-based control diet, indicating that Oralject™ induces the augmented uptake of intact bioactive proteins.

Oral Vaccine Delivery

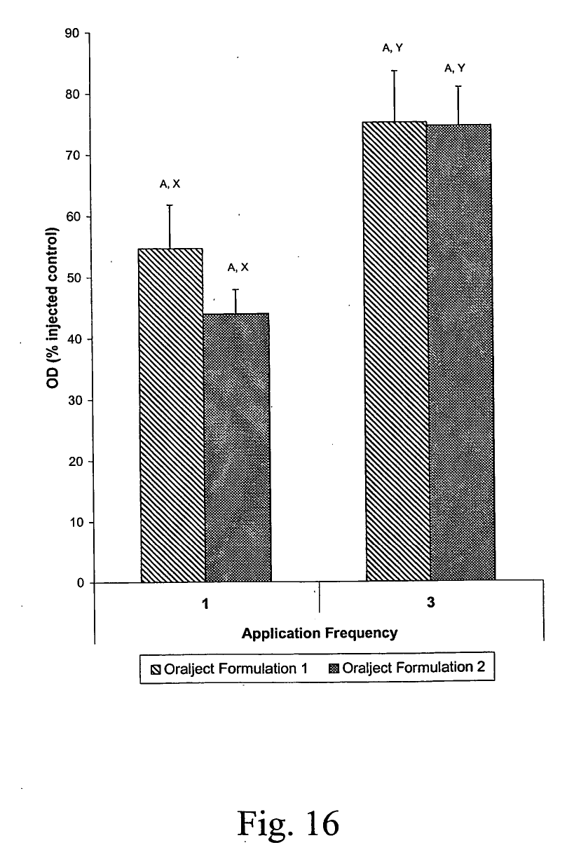

FIG. 16 shows the influence of Oralject™ formulation type and application frequency on antibody level (expressed as a percentage of OD versus serum pool from selected high-titre injected controls). There was no influence of Oralject™ formulation type, however, application frequency significantly affected antibody level, with multiple applications providing increased antibody levels.

In FIG. 16, relative antibody levels (% OD of injected control at serum dilution of 1:100) 4 weeks following the start of the experiment, in rainbow trout following oral administration of A. salmonicida vaccine using different (a,b Within Application Frequency, different superscripts are significantly different (P<0.05), x, y Within Oralject Formulation, different superscripts are significantly different (P<0.05).

### EXAMPLE VI

### Large-Scale Oral Vaccine Delivery

Materials and Methods

An external oral vaccination study was conducted at the Resource and Productivity Council, Fredericton NB, using an adaptation of the bypass cocktail described in table 1. Brook trout (Salvelinus fontinalis; n=1600, average mass 65 g) were divided into 8 tanks (flow-through system with dechlorinated city water, 11° C.). Duplicate groups were fed a lyophilized commercial Furunculosis vaccine (ALPharma) in either the Oralject™ System (OR) or in a commercial feed matrix (FA). Positive control fish were injected with vaccine (0.2 ml; ip), whereas negative controls were untreated. Oral treatments were repeated (booster application) at 250 degree-days following initial inoculation. Serum was collected at bi-weekly intervals and in vivo cohabitation challenge was initiated at 450 degree-days following initial inoculation.

Results

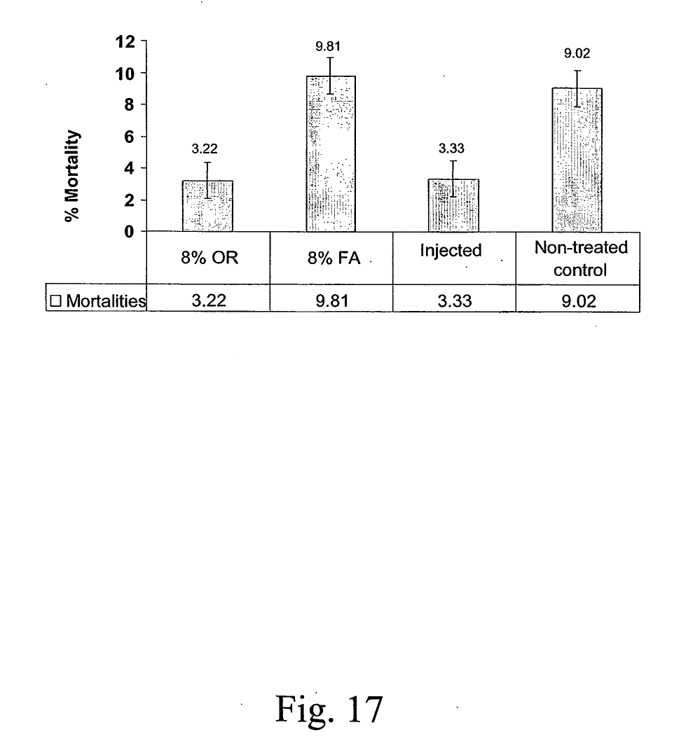

FIG. 17 clearly demonstrates that fish treated orally with the Oralject™-vaccine is as effective as the injection treatment to protect animals against infection to Aeromonas salmonicida. Moreover, antibody titres were higher in the Oralject™ group than those observed in non-treated group or in fish fed with commercial feed containing the same concentration of vaccine (results not shown).

### EXAMPLE VII

### Large-Scale Oral Vaccine Delivery

In this study, we undertake to evaluate the efficiency of a furunculosis vaccine administrated with the oral delivery system Oralject™, which is an adaptation of the bypass cocktail described in table 1. Thus, vaccine was administered using the pelleted OralJect™ system and both, humoral response and the protection against Aeromonas salmonicida were monitored and compared with the injected vaccine.

Materials and Methods

Brook trout, initial mass 88 g, 185 fish/group.

| | |
|---|---|
| INJ | AS Vaccine Intraperitoneal Injection |
| OR8 | Oralject ™ + AS Vaccine (8% w:w) (one dose) |
| CTRL | Untreated |

On day 28, all treated groups received a booster (B) dose of vaccine treatment.
Vaccination Procedures

As recommended by supplier, injected fishes (INJ) received 0.2 ml of PBS containing with 1.25% (w/v) of furunculosis vaccine (Alpharma, Oslo, Norway) for both, the first injection and the booster injection. Booster injection was administrated at day 28.

For the first immunization, fishes from fish groups or Oralject groups received 1% of its wet weight administrated orally. The second vaccination was performed the same way 28 days following first vaccination. Fish from control group (CTRL) were untreated.

Schedule

| | |
|---|---|
| Day 0 | First immunization |
| Day 14 | Antibody dosage |
| Day 28 | Second immunization (boost; all groups), Antibody |
| | dosage |
| Day 42 | Antibody dosage |
| Day 70 | Antibody dosage, Challenge (Co-habitation) |
| Day 84 | Report |

Dosage of Antibody Response

Antibody response was analyzed by ELISA, which is routinely used in our laboratory.

Challenge Tests

For the in vivo challenge methodology, fish were challenged by cohabitation. This method consists of placing previously infected fish with the treated fish and following the mortality to a pre-determined limit, a given percentage (30-40% according to the literature) of control fish mortality.

Results

Antibody Response

Peak of antibody response was reached 6 weeks following first immunization in all treatments.

Challenge

During the challenge, fish were challenged by cohabitation. Although antibody levels were almost the same as untreated level except the injected treatments, protection against occurred.

TABLE 2
Cohabitation challenge results

| | | Mortality | |
|---|---|---|---|
| Treatments | Mortalities | percentage | RPS |
| INJ-B | 7/159 | 4.4 | 89.2 |
| OR8-B | 13/164 | 7.9 | 80.5 |
| CTRL | 58/180 | 40.6 | 0 |

Relative Percentage of Survival, RPS: (1 − % Mortality treated/% Mortality untreated) × 100

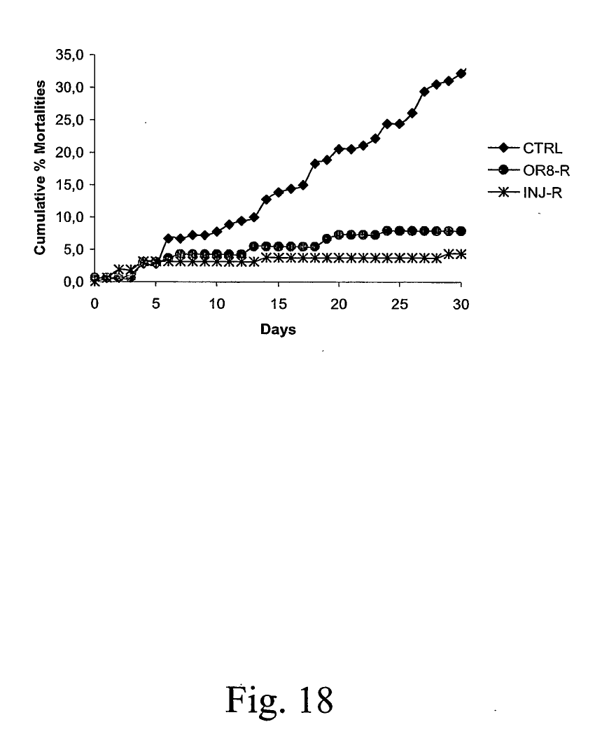

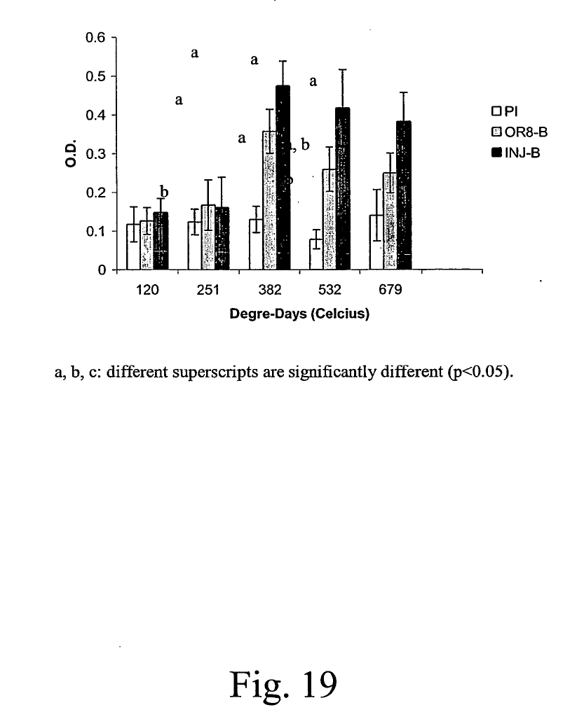

FIG. 18 clearly demonstrates that fish treated orally with the Oralject™-vaccine is as effective as the injection treatment to protect animals against infection to Aeromonas salmonicida following cohabitation challenge. Moreover, antibody titres were higher in the Oralject™ group than those observed in non-treated group or in fish fed with commercial feed containing the same concentration of vaccine (FIG. 19).

### Conclusion

Bioactive proteins (in this case, HRP) can be delivered orally to rainbow trout using the Oralject™ System, with subsequent appearance of intact protein in the general circulation. Using this strategy, approximately 18-fold increase in circulating HRP was measured versus oral delivery in a fish meal based formulation. Rainbow trout fed A. salmonidicida vaccine in the Oralject System show antibody levels up to 75% of that from injected control fish. Also, these results suggest that Oralject™ treatment provides a similar protection than injected methodology.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition for modulating a physiological reaction or inducing an immune response in human or animal after oral administration, said composition comprising:
a) at least one physiologically active agent;
b) at least one neutralizing agent effective to increase pH in digestive system of said human of animal to prevent denaturation of said physiologically active agent;
c) at least one inhibitor of digestive enzymes to prevent enzymatic digestion of said physiologically active agent, said inhibitor being selected from the group consisting of homogenized legumes, oilseed or pulse grains; and
d) at least one uptake-increasing agent capable of increasing intestinal absorption of said physiologically active agent.

2. The composition of claim 1, wherein said neutralizing agent is at concentration between 1% to 60% w/w, said inhibitor is at concentration between 1% to 50% w/w, and said uptake increasing agent is at concentration between 0.1% to 50% w/w.

3. The composition of claim 1, wherein said physiologically active agent is selected from the group consisting of therapeutical agents, nutritional products, mucopolysaccharides, lipids, carbohydrates, steroids, hormones, growth hormones (GH), growth hormone releasing hormones (GHRH), epithelial growth factors, vascular endothelial growth and permeability factors (VEGPF), nerve growth factors, cytokines, interleukins, interferons, GMCSF, hormone-like products, neurological factors, neurotropic factors, neurotransmitters, neuromodulators, enzymes, antibodies, peptides, proteic fragments, vaccines, adjuvants, antigens, immune stimulating or inhibiting factors, heomatopoietic factors, anti-cancer products, anti-inflammatory agents, anti-parasitic compounds, anti-microbial agents, nucleic acid fragments, plasmid DNA vectors, cell proliferation inhibitors or activators, cell differentiating factors, blood coagulation factors, immunoglobulins, negative selective markers or “suicide” agents, toxic compounds, anti-angiogenic agents, polypeptides, anti-cancer agents, acid production drugs, and histamine H2-receptor antagonists.

4. The composition of claim 1, wherein said neutralizing agent is in an amount sufficient to neutralize acidic degradation in said human or animal digestive system and to allow delivery of said physiologically active agent to intestine of said human or animal.

5. The composition of claim 1, wherein said neutralizing agent is selected from the group consisting of anti-acids, sodium bicarbonate, sodium carbonate, sodium citrate, calcium phosphate, calcium carbonate, magnesium salts, magnesium carbonate, magnesium trisilicate, magnesium hydroxide, magnesium phosphate, magnesium oxide, bismuth subcarbonate, and combinations thereof.

6. The composition of claim 1, wherein said neutralizing agent is at least one of sodium carbonate at a concentration of 10% to 20% w/w, and calcium carbonate at concentration of 10% to 20% w/w of the composition.

7. The composition of claim 1, wherein said inhibitor is in an amount sufficient to inhibit degradation of said physiologically active agent by digestive enzymes in said human or animal digestive system and to allow delivery of said physiologically active agent to intestine of said human or animal.

8. The composition of claim 1, wherein said inhibitor of digestive enzymes is selected from the group consisting of anti-protease, egg albumin, plant-derived inhibitors from oilseed, soybean, kidney bean, faba bean, rice bran, wheat bran, ethylenediamine tetraacetate, alpha-1-antitrypsin, albumin, ovalbumin, and proteosomes.

9. The composition of claim 1, wherein said inhibitor comprises at least one of a pepsin inhibitor and an enteropeptidase inhibitor.

10. The composition of claim 1, wherein said inhibitor is albumin at a concentration between 1% to 20% w/w.

11. The composition of claim 1, wherein said uptake increasing agent is selected from the group consisting of a bile salt, saponin, deoxycholate, sodium salicylate, sodium lauryl sulphate, oleic acid, linoleic acid, monoolein, lecithin, lysolecithin, polyoxyethylene sorbitan ester, p-t-octylphenoxypolyoxyethylene, N-lauryl-β-D-maltopyranoside, 1-dodecylazacycloheptane-2-azone, and phospholipid.

12. The composition of claim 11, wherein said uptake-increasing agent is deoxycholate at a concentration between 0.01% to 10%.

13. The composition of claim 1 comprising at least one additional ingredient selected from the group consisting of ethylenediamine tetraacetate, a preservative, an antioxidant, a colorant, a binder, a tracer, a sweetener, a surfactant, a unmoulding agent, a flavouring agent, meal, bean, yeast, brewer yeast, mineral oil, vegetable oil, animal oil, a lubricant, an ointment, and combinations thereof.

14. The composition of claim 1, wherein said physiologically active agent when delivered in intestine of said human or animal is absorbed by said intestine for systemic delivery.

15. The composition of claim 1, wherein said physiologically active agent when delivered in intestine of said human or animal has an effective physiological effect on intestinal wall.

16. The composition of claim 1, wherein said physiologically active agent when delivered in intestine of said human or animal has a physiological effect on the content of the intestine.

17. The composition of claim 1, wherein said animal is a bird, a mammal, an insect, a crustacean, an amphibian, a reptile or a fish.

18. The composition of claim 1, wherein said physiologically active agent is capable of inducing an immune response in said human or animal against mucosal infectious diseases.

19. The composition of claim 1, wherein said modulating comprises increasing or reducing the rate of a physiological reaction.

20. A method for modulating a physiological reaction or inducing an immune response in a human or an animal comprising orally administering to said human or animal a sufficient amount of a composition as defined in claim 1.

21. The method of claim 20, wherein said physiological reaction is at least one of body growth, immune reaction, fat metabolism, or muscle synthesis.

22. A method of systemic delivery of a physiologically active agent to a human or an animal, said method comprising orally administering to said human or animal a composition as defined in claim 1.

23. A method for enhancing body uptake of a physiologically active agent or an antigen in a human or an animal comprising orally administrating to said human or animal a physiologically effective amount of a composition as defined in claim 1.

24. Use of a composition according to claim 1 in the manufacture of a drug or a food for modulating a physiological reaction or inducing an immune response in human or animal.
